(19) United States
(12) Patent Application Publication
(10) Pub. No.: US 2004/0224907 A1
(43) Pub. Date: Nov. 11, 2004

(54) Compositions and methods for reversal of drug resistance

(76) Inventors: Gavril W. Pasternak, New York, NY (US); Claire Neilan, Newark (DE)

Correspondence Address: FROMMER LAWRENCE & HAUG, 745 FIFTH AVENUE- 10TH FL., NEW YORK, NY, 10151, US

(21) Appl. No.: 10/827,063

(22) Filed: Apr. 19, 2004

Related U.S. Application Data
(63) Continuation-in-part of application No. PCT/US02/33551, filed on Oct. 21, 2002.
(60) Provisional application No. 60/336,434, filed on Oct. 19, 2001.

(51) Int. Cl. A61K 31/704; A61K 31/537; A61K 31/55; A61K 31/522

(52) U.S. Cl. 514/34; 514/217.12; 514/237.5; 514/408; 514/449

(57) ABSTRACT

The present invention relates to sigma-1 receptor ligands, to uses of sigma-1 receptor ligands in treating drug resistance, methods of using sigma-1 receptor ligands in regulating P-glycoprotein expression, methods of screening for compositions (e.g., agonists) that activate the sigma-1 receptor, regulate P-glycoprotein expression and/or have tolerance reducing activity.

### RELATED APPLICATIONS/PATENTS & INCORPORATION BY REFERENCE

This is a continuation-in-part application of International Application Number PCT/US02/33551, filed on Oct. 21, 2002, which claims priority to U.S. Provisional Application No. 60/336,434, filed Oct. 19, 2001, the contents of which are expressly incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein cited references”), as well as each document or reference cited in each of the herein-cited references (including any manufacturer's specifications, instructions, etc.), is hereby expressly incorporated herein by reference.

### STATEMENT OF RIGHTS TO INVENTION MADE UNDER FEDERALLY SPONSORED RESEARCH

This work was supported by the government, in part, by a grant from the National Institute on Drug Abuse (DA6241). The government may have certain rights to this invention.

### TECHNICAL FIELD

The present invention relates to sigma-1 receptor ligands, to uses of sigma-1 receptor ligands in treating drug resistance, methods of using sigma-1 receptor ligands in regulating P-glycoprotein expression, methods of screening for compositions (e.g., agonists) that activate the sigma-1 receptor and regulate P-glycoprotein expression.

### BACKGROUND ART

Multidrug resistance (MDR) is a major obstacle in the successful treatment of cancer. Neoplastic cells often develop pleiotropic resistance to unrelated anticancer therapies after initial treatment, thereby propagating recurrence of the disease. A hallmark of the MDR phenotype is increased expression of a family of ATP-dependent transmembrane pumps collectively known as ATP-binding cassette (ABC) transporters. One in particular, known as P-glycoprotein (Pgp), is frequently expressed at high levels in many tumor cells. Other proteins implicated in MDR are multi-drug resistance-associated protein (MRP), lung resistance-related protein (LRP), and breast cancer resistance protein (BCRP) (Tan, et al. (2000) Curr. Opin. Oncol. 12(5): 450-8). The mechanism of action of these ABC transporters is to facilitate efflux of natural xenobiotics out of the cell. In the case of tumor cells, high levels of Pgp and its related family members cause transport of chemotherapeutics out of the tumor cell, thereby preventing the intracellular accumulation of the drug from its target. Thus, there is a strong correlation between expression of Pgp and the MDR phenotype (Scotto et al. (1986) Science 232(4751): 751-5).

Pgp is encoded by the MDR1 gene and is a large (170 kD) transmembrane protein. Pgp is expressed in many cell types, including but not limited to: adrenals, blood-brain barrier, liver, large intestine, and kidney. Upregulation of steady-state Pgp levels appears to be a default mechanism of the stress response pathway, and its expression is regulated at the messenger RNA level (Shtil, A. A. (2001) Curr. Drug Targets 2(1): 57-77). The Pgp protein contains two nucleotide binding motifs, and is both phosphorylated and glycosylated. Nucleotide binding and the aforementioned post-translational modifications are necessary for full activity (Kramer et al, (1995) Br. J. Cancer 71(4): 670-5; Fine et al, (1996) Oncologist 1(4): 261-268; van Den Elsen et al, (1999) Proc. Natl. Acad. Sci. 96(24): 13679-84). Related to drug efflux, Pgp is implicated in altering membrane fluidity by directly impinging on cholesterol levels in membrane domains (Garrigues et al, (2002) Proc. Natl. Acad. Sci. USA 99(16): 10347-52). Additionally, Pgp may also alter the phospholipid content between the extracellular and intracellular leaflets (Romsicki et al, (2001) Biochemistry 40(23): 6937-47). This may directly contribute to its mechanism of efflux.

Under normal physiological conditions, Pgp confers natural resistance to xenobiotics. However, Pgp is highly expressed in a number of solid tumors, such as gliomas, non-small cell lung cancer, renal cell carcinoma, and colon cancer (Ross, D. D. (2000) Leukemia 14: 467-473). As a result of Pgp overexpression, these tumors are resistant to various, structurally unrelated anticancer agents, including, but not limited to anthracyclines, vinca alkaloids, epipodophyllotoxins, and taxanes. Drugs that interact with Pgp are frequently large, hydrophobic or amphipathic molecules with a planar ring system and a basic nitrogen side chain (Pearce et al. (1989) Proc. Natl. Acad. Sci. USA 86: 5128-32). This preference for these molecules may be a result of the hypothesized role Pgp plays in altering membrane fluidity and phospholipid content. Many anticancer agents are, in fact, substrates for Pgp, and as a result, cancers that are initially sensitive to these agents often become resistant after initial treatment. Inhibition of Pgp can occur at many different levels, such as, but not limited to direct inhibition of the Pgp mechanism of action (efflux), inhibition of Pgp modifiers, and inhibition of Pgp expression.

Despite its attractiveness as a target for drug therapy, Pgp inhibition has been clinically unsuccessful thus far. Numerous compounds have been used to directly inhibit Pgp's mechanism of action, such as verapamil, cyclosporin A, and analogs thereof. Interactions between these drugs and chemotherapeutic agents contributing to tumor cell cytotoxicity have been well characterized. Inhibition of Pgp mechanism of action falls into four major categories: 1) non-competitive inhibition, 2) competitive inhibition, 3) allosteric inhibition, and 4) cooperative stimulation, suggesting that the substrate binding behavior of the various ligands occurs at more than one binding site on Pgp (Litman et al, (1997) Biochim. Biophys. Acta 136(2): 169-76). That said, the most well characterized Pgp inhibitors, verapamil and cyclosporin A, often have pleiotropic effects when combined with chemotherapeutic agents, often affecting pharmacokinetics and elimination of the anticancer drug (Sikic et al, (1997) Cancer Chemother. Pharmacol. 40 (Suppl.): S13-S19).

Verapamil is typically used as a treatment for cardiac arrhythmias and hypertension, and acts as a calcium channel blocker. It acts as a competitive inhibitor of Pgp and as an allosteric inhibitor in the presence of other anticancer compounds (Sikic et al, (1997) Cancer Chemother. Pharmacol. 40 (Suppl.): S13-S 19). However, the dosages necessary for verapamil to effectively block Pgp efflux causes cardiotoxicity and abnormal ion channel function. The same holds true for cyclosporin A (CsA), an immunosuppressant primarily used to prevent rejection from organ transplantation and other autoimmune-related conditions, such as psoriasis. At high doses, CsA has been known to cause nephrotoxicity. Additionally, its immunosuppressive effects are contraindicated during anticancer therapy. Thus, “first-generation” Pgp inhibitors precluded clinical use in anticancer therapy. Many other Pgp inhibitors have been developed or discovered, such as anti-malarials, cardiovascular drugs, certain antibiotics, and phenothiazines. Most have uses in the treatment of other diseases and like verapamil and CsA, have other unwanted effects at the doses necessary to inhibit PgP.

Another approach is to target post-translational modifications that are required for Pgp activity. Pgp contains numerous phosphorylation sites and interestingly, the MDR phenotype and phosphorylation of Pgp were diminished in the presence of staurosporine, an inhibitor of protein kinase C (PKC) (Laredo et al, (1994) Blood 84(1): 229-37). However, mutation of all phosphorylation sites in Pgp did not diminish activity (Conseil et al, (2001) Biochemistry 40(8): 2564-71). Another post-translational modification required for Pgp activity is glycosylation. Eliminating carbohydrate groups from Pgp by tunicamycin has been tested ex vivo only (Kramer et al, (1995) Br. J. Cancer 71(4): 670-5), and as tunicamycin is not specific for Pgp glycosylation, pleiotropic effects would preclude its clinical use.

Other approaches to inhibit Pgp have been considered, such as the inhibition of Pgp expression at the messenger RNA level and/or at the protein level. One such method is through the use of antisense oligonucleotides to block transcription of Pgp (mdr1) mRNA (Pan et al, (2001) Chin. Med. J. 114(9): 929-32). However, the use of mdr1 antisense oligonucleotides in a clinical setting has not been established, as experiments were performed only in ovarian carcinoma cell lines ex vivo. Preliminary work has also implicated the use of monoclonal antibodies via liposomal delivery as a method for Pgp inhibition, but as with the use of antisense technology, these antibodies were not used in vivo and its clinical relevance is unknown (Matsuo et al, (2001) J. Control Release 77(1-2):77-86). Another antibody used in detection of Pgp, C219, has been shown to bind both ATP binding domains and diminishes both its ATPase and drug binding capacities (van Den Elsen et al, (1999) Proc. Natl. Acad. Sci. 96(24): 13679-84). However, use of this antibody in a clinical setting is not recommended as it recognizes epitopes on other proteins such as heavy chain muscle myosin, among others (Schinkel et al, (1991) Cancer Res. 51(10): 2628-35, Thiebaut F, et al J. Histochem. Cytochem. 37(2): 159-64).

An ideal Pgp inhibitor would be a substance that allows downregulation of Pgp expression at either the mRNA or protein level, which would decrease the unwanted effects that other previously characterized Pgp inhibitors exhibit in conjunction with standard anticancer therapies. Simply blocking efflux by Pgp provides only a temporary solution, to which the tumor cell can respond to by increasing Pgp expression. Directly impinging on Pgp expression in the tumor cell, therefore, would decrease the steady-state levels of available Pgp and allow chemotherapeutic agents to reach their target cells in effective concentrations.

Investigations into possible inhibitors of Pgp have led to studies involving the sigma receptor. Sigma receptors are localized to the endoplasmic reticulum and are of low molecular weight, consisting of one to two transmembrane domains. Two isoforms, named sigma-1 and sigma-2, are found in many different tissues in the body, such as but not limited to, the central nervous system, lymphoid, reproductive, and endocrine tissues. Sigma receptors are also found at high density in tumor cells (Vilner B J, et al (1995) Cancer Res. 55(2): 408-13, Brent P J, et al (1995) Eur. J. Pharmacol. 278(2): 151-60). Sigma receptors have been implicated in modulation of glutamergic function via the N-methyl-D-aspartate receptor complex, tonic potassium channel function, intracellular calcium levels, analgesic effects of opioids, and neuroleptic responses. Sigma receptors bind a wide variety of chemically unrelated drugs, some of which elicit psychomimetic effects, such as opioids, neuroleptics, and dopaminergic phenylpiperidine analogs. Sigma sites display high affinity for aromatic ring structures with an amine nitrogen. Compounds with the highest sigma receptor affinities include benzomorphans and the anti-psychotic butyrophenone, haloperidol (Largent B L, et al (1987) Mol. Pharmacol. 32(6): 772-84).

Sigma-1 and sigma-2 isoforms exhibit specificity for different ligands and selectivity between different stereoisomers of the same ligand. For example, sigma-1 receptors have increased selectivity for (+)enantiomers like (+)pentazocine and (+)SKF 10047, while sigma-2 receptors show reverse stereoselectivity (Hellewell S B, et al (1990) Brain Res. 527: 224-253, Georg A, et al (1991) J. Pharmacol. Exp. Ther. 259: 479-83). Sigma-2 receptors have little, if any, affinity for (+)pentazocine. It is unknown whether the receptors share any similarity on a molecular level. In general, the properties of the sigma-2 receptor are not well characterized.

Given its high receptor density in tumor cell lines, various sigma ligands have been implicated for use in diagnostic imaging (Vilner B J, et al (1995) Cancer Res. 55(2): 408-13). In addition to diagnostic use, studies have also demonstrated that use of sigma ligands such as the benzomorphans have a direct effect on tumor cell growth. Brent and coworkers showed that administration of sigma ligands directly impinges on ex vivo growth of tumor cells derived from human mammary adenocarcinoma, colon carcinoma, and melanoma (Brent P J, et al (1995) Eur. J. Pharmacol. 278(2): 151-60), although it is unknown if these tumor cell lines were multidrug resistant. Furthermore, work by Callaghan et al. demonstrated that natural and synthetic opiates such as morphine, pentazocine, and meperidine were able to interact directly with Pgp in multidrug resistant cells. Not only were these ligands able to displace iodomycin from Pgp, but they were also able to increase intracellular accumulation of vinblastine in resistant cells (Callaghan R, et al (1993) J. Biol. Chem. 268(21): 16059-64). This collateral sensitivity of Pgp expressing cell lines to opiate derivatives was attributed to increased membrane fluidity and loss of basal levels of Pgp phosphorylation, not through modulation of sigma receptors or by down-regulation of Pgp expression levels (Callaghan R, et al (1995) Biochim. Biophys. Acta 1236(1): 155-62). However, upon administration of sigma-2 agonists, Pgp mRNA levels were decreased, the potency of DNA damaging agents were enhanced, and apoptosis was induced in drug-resistant cancer cells (Bowen W D, (2000) Pharm. Acta Helv. 74(2-3): 211-18). It is currently unknown whether sigma-1 agonists have similar effects on multidrug resistant cells. At least one report suggests that sigma-1 agonists do not effect Pgp mRNA expression levels (Bowen W D, et al (1997) Proceedings of the Eighty-Eighth Annual Meeting of the American Association for Cancer Research, Vol. 38). Bowen et al. reported that treatment of human neuroblastoma cells with BD 737, which binds to both sigma-1 and sigma-2 receptors, had no significant effect on expression of the mdr-1 gene (the Pgp gene is also known as the mdr-1 gene). Bowen et al. also reported that specific activation of sigma-2 receptors resulted in decreased mdr-1 gene expression, thereby demonstrating the “opposing effects of sigma-1 and sigma-2 receptor activation.” (Bowen W D, et al (1997)).

Investigations have heretofore yielded peripheral evidence linking sigma-2 receptor ligands to the treatment of drug resistant cancers. However, direct evidence linking sigma-1 ligands and the sigma-1 receptor to down-regulation of Pgp expression, and hence, drug resistance, was heretofore unknown.

### OBJECT AND SUMMARY OF THE INVENTION

It has now surprisingly been shown that binding of a sigma-1 ligand to the sigma-1 receptor restores drug sensitivity in drug resistant cells. For example, treatment of drug resistant cells with a sigma-1 ligand (e.g., (+)pentazocine) down-regulates expression levels of Pgp at the mRNA and protein levels in cells, thereby sensitizing these cells to chemotherapeutics. Use of sigma-1 ligands to restore drug sensitivity (e.g., chemotherapeutic sensitivity) will yield substantial clinical results. Furthermore, screens to search for agents, including novel sigma-1 receptor ligands, which can be used in reversal of the MDR phenotype are now envisioned.

Accordingly, in one embodiment, the present invention relates to a method of treating a drug resistant phenotype comprising administering a sigma-1 receptor ligand to a subject in an amount sufficient to restore drug sensitivity.

In yet another embodiment, the present invention relates to a method for reducing or ameliorating a drug resistant phenotype ex vivo comprising treating a cultured cell with a sigma-1 receptor ligand in an amount sufficient to restore drug sensitivity.

In yet another embodiment, the present invention relates to a method of reducing P-glycoprotein expression in a cell, wherein the method comprises the steps of:

1) contacting the cell with a sigma-1 receptor ligand;

2) binding the sigma-1 receptor ligand to the sigma-1 receptor;

3) reducing P-glycoprotein expression in the cell.

In yet another embodiment, the present invention relates to a method of screening compositions for tolerance-reducing activity, wherein the method comprises the steps of:

1) contacting the test cell with a composition potentially comprising a sigma-1 receptor ligand;

2) separately measuring the levels of P-glycoprotein expression in an untreated control cell and test cell;

3) detecting a reduction in P-glycoprotein expression in the test cell; and

4) comparing P-glycoprotein expression in the test cell to the control cell.

In yet another embodiment, the present invention relates to a method of screening agents for sigma-1 receptor binding activity, wherein the method comprises the steps of:

1) contacting a potential sigma-1 receptor ligand test agent with a test cell that expresses the sigma-1 receptor and high levels of P-glycoprotein;

2) binding the test agent to the sigma-1 receptor; and

3) detecting a reduction in P-glycoprotein expression in the test cell.

In yet another embodiment, the present invention relates to a method of screening compositions for tolerance-reducing activity, wherein the method comprises the steps of:

1) treating a control chemotherapeutic-sensitive cell and a chemotherapeutic-resistant test cell with said chemotherapeutic agent;

2) contacting the test cell with a composition potentially comprising a sigma-1 receptor ligand;

3) separately measuring the level of chemotherapeutic sensitivity in the control cell and test cell; and

4) detecting an increase in sensitivity in the test cell.

These and other objects and embodiments are described in or are obvious from and within the scope of the invention, from the following Detailed Description.

### BRIEF DESCRIPTION OF THE DRAWINGS

FIG. 1. depicts the time dependent reduction in Pgp mRNA levels following 10 μM (+)pentazocine treatment. RT-PCR was performed and [³²P]ATP was incorporated into the PCR reaction. Radioactivity was measured following gel separation of PCR product.

FIG. 2. depicts dose-dependent reduction of Pgp mRNA levels following (+)pentazocine treatment. RT-PCR was performed and [³²P]ATP was incorporated into the PCR reaction. Radioactivity was measured following gel separation of PCR product.

FIG. 3. depicts Western Blot showing time-dependent reduction of Pgp expression in BE(2)—C cells following 10 μM (+)pentazocine treatment.

FIG. 4. depicts Western blot showing time-dependent reduction of Pgp expression in ADX cells following 10 μM (+)pentazocine treatment.

### DETAILED DESCRIPTION OF THE INVENTION

In one aspect, the present invention is directed to a method of treating a drug resistant phenotype manifested in a subject comprising administering a sigma-1 receptor ligand to the subject in an amount sufficient to reverse drug resistance.

The following terms shall have the meaning set forth below:

A “subject” is a vertebrate, preferably a mammal, and more preferably a human. Mammals include, but are not limited to, humans, farm animals, sport animals, and pets.

An amount sufficient to reverse drug resistance is any therapeutically effective amount.

A “therapeutically effective amount” is an amount sufficient to effect a beneficial or preferably, desired clinical result (e.g., improved or restored drug sensitivity). A therapeutically effective amount can be administered in one or more doses. In terms of treatment, an effective amount is an amount that is sufficient to palliate, ameliorate, reverse, or slow the progression of, or otherwise reduce the associated pathological consequences (e.g., drug resistance). A therapeutically effective amount can be provided in one or a series of administrations (e.g., divided doses) and is generally determined by the physician on a case-by-case basis, a determination that is well within the skill of one in the art.

As a rule, the dosage for in vivo therapeutics or diagnostics will vary. Several factors are typically taken into account when determining an appropriate dosage. These factors include age, sex and weight of the patient, the condition being treated, the severity of the condition and the form of the agent being administered.

A “sigma-1 receptor ligand” is an agonist, or partial agonist, that binds to the sigma-1 receptor and results, directly or indirectly, in decreased P-glycoprotein expression. Sigma-1 receptor ligands of the invention can be in the form of a (+)enantiomer. Sigma-1 receptor ligands include, but are not limited to, pentazocine, (+)pentazocine, (+)N-allylnormetazocine, 2-(4-morpholinethyl)-1-phenylcyclohexanecarboxylate, cis-N-[2-(3,4-dichlorophenyl)ethyl]-N-methyl-2-(1-pyrrolidinyl)cyclohexylamine, and N-[2-3,4-dichlorophenyl)ethyl]-N-methyl-2-(1-azapinyl)ethylamine dihydrochloride, but most preferably, (+)pentazocine. Sigma-I receptor ligands of the present invention, including, but not limited to, 2-(4-morpholinethyl)-1-phenylcyclohexanecarboxylate and cis-N-[2-(3,4-dichlorophenyl)ethyl]-N-methyl-2-(1-pyrrolidinyl)cyclohexylamine, can bind to both sigma-1 and sigma-2 receptors, thereby decreasing P-glycoprotein expression. A sigma-1 receptor ligand of the present invention can also bind exclusively to the sigma-1 receptor. Dosing and administration schedules for Sigma-1 receptor ligands can be as indicated in published texts, such as the Physican's Desk Reference and Goodman and Gillman's The Pharmacological Basis for Therapeutics, 10th Ed. (2001), McGraw-Hill Companies, Inc.

Sigma-1 receptor ligands such as pentazocine can be administered in an oral range that is preferably up to 300 mg every 3-4 hours. Doses can range from about 5-10 mg, 10-25 mg, 25-50 mg, 50-100, 100-200 mg, up to about 300 mg.

In one embodiment, methods of the present invention are carried out by administering to a subject at least one sigma-1 receptor ligand, such that binding of the sigma-1 receptor ligand to the sigma-1 receptor in drug resistant cells reduces P-glycoprotein expression. Preferably, in a subject undergoing a drug treatment regimen, pleiotropic drug sensitivity (e.g., multidrug sensitivity) increases in a population of drug resistant cells after, or concurrent with, the administration of at least one sigma-1 receptor ligand.

In yet another aspect, the present invention is directed to a method of preventing or reducing a drug resistant phenotype in a subject comprising administering a sigma-1 receptor ligand to the subject in an amount sufficient to enhance drug sensitivity and/or prevent drug resistance prior to the onset of drug resistance. Preferably, in a subject who will undergo a drug treatment regimen, pleiotropic drug sensitivity (e.g., multidrug sensitivity) increases in a population of potentially drug resistant cells after, or concurrent with, the administration of at least one sigma-1 receptor ligand.

Accordingly, in one embodiment, methods of the present invention are carried out by first administering to a subject at least one sigma-1 receptor ligand, such that binding of the sigma-1 receptor ligand to the sigma-1 receptor in potentially drug resistant cells reduces, stabilizes or otherwise beneficially modulates P-glycoprotein expression. Preferably, pleiotropic drug sensitivity is reduced or avoided in a population of potentially drug resistant cells subject to prior treatment with at least one sigma-1 receptor ligand.

Administration can be by all known routes, including, but not limited to, oral, topical, subcutaneous, intramuscular, intrathecal, injectable, intravenous and inhaled routes of administration.

Sigma-I receptor ligands of the present invention, including those identified by the methods described herein, can be administered in formulations comprising a pharmaceutically acceptable excipient, such as water, saline, aqueous dextrose, glycerol, or ethanol. Although it is not crucial, dilution and/or formulation of the sigma-1 receptor ligand in a physiologically acceptable excipient can be important and useful in providing the final dosage concentration.

The compositions can be supplied in solid, semi-solid or liquid forms, including tablets, capsules, powders, liquids, sprays and suspensions. Aqueous suspensions can contain the composition in admixture with pharmaceutically acceptable excipients such as suspending agents, e.g., sodium carboxymethyl cellulose, methylcellulose, hydroxypropylmethylcellulose, sodium alginate, polyvinylpyrrolidone, gum tragacanth and gum acacia; dispersing or wetting agents such as naturally occuring phosphatide, e.g., lecithin, or condensation products of an alkylene oxide with fatty acids, e.g., polyoxyethylene stearate, or condensation products of ethylene oxide with long chain aliphatic alcohols, e.g. heptadecaethyleneoxycetanol, or condensation products ethylene oxide with partial esters derived from fatty acids and a hexitol, e.g. polyoxyethylene sorbitol monoleate or condensation products of ethylene oxide with partial esters derived from fatty acids and hexitol anhydrides, e.g., polyoxyethylenes sorbitan monooleate. Such aqueous suspensions can also contain one or more preservatives, e.g., ethyl or n-propyl-p-hydroxy benzoate.

Dispersible powders and granules suitable for preparation of an aqueous suspension by the addition of water provide the composition in admixture with a dispersing or wetting agent, suspending agent and one or more preservatives. Suitable dispersing or wetting agents and suspending agents are exemplified by those already mentioned above. The invention thus encompasses concentrated forms for subsequent dilution before use or sale. Also contemplated by this invention are slow-release or sustained-release forms, whereby a relatively consistent level of the sigma-1 receptor ligands are provided over an extended period. The compositions can further comprise other medicinal agents, including, but not limited to, pharmaceutical agents (e.g., chemotherapeutic agents), adjuvants, carriers, and auxiliary substances, such as wetting or emulsifying agents, and pH buffering agents.

Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate formulations is within the skill of one in the art given the parameters herein.

In several embodiments, a subject is undergoing, has undergone, or will undergo drug treatment with one or more drugs to which resistance can develop. Preferably, the drugs comprise chemotherapeutic agents, including but not limited to, actinomycin D, doxorubicin, mitoxantrone, paclitaxel and vincristine, but most preferably, paclitaxel or doxrubicin. Preferably, the subject is undergoing, has undergone or will undergo treatment with the chemotherapeutic agent doxorubicin and is treated with the sigma-1 receptor ligand, (+)pentazocine. In yet another preferred embodiment, the subject is undergoing, has undergone or will undergo treatment with the chemotherapeutic agent paclitaxel, and is treated with the sigma-1 receptor ligand, (+)pentazocine.

In yet another aspect of the present invention, cultured cells of a subject can be treated with a sigma-1 receptor ligand in an amount sufficient to prevent, reduce or ameliorate a drug resistant phenotype ex vivo. For example, cells of this embodiment can be undergoing unwanted proliferation (e.g. malignant or benign cells, such as tumor cells) and have developed resistance to one or more drugs, preferably chemotherapeutic agents. As another example, cells of this embodiment can be stem cells treated with a sigma-1 receptor ligand to prevent the development of drug resistance upon transplantation into a subject undergoing, or about to undergo, drug therapy (e.g., chemotherapy). Cultured cells of this embodiment can be obtained from any tissue source, including a tissue selected from the group consisting of brain, uterine, blood, breast, thyroid, pancreas, gastroinstestinal, ovarian, prostate, lung, skin and lymphatic tissue.

In yet another aspect, the present invention relates to methods of screening compositions for tolerance-reducing activity (e.g., screening for sigma-1 receptor agonists). “Tolerance-reducing activity” is one or more actions by an agent that reduces drug tolerance. “Drug tolerance” comprises, for example, drug resistance, preferably, multidrug resistance.

In one embodiment, methods of screening compositions for tolerance-reducing activity are carried out by first obtaining a control cell that expresses high levels of P-glycoprotein, and obtaining a test cell that is the same as the control cell. Next, the test cell is contacted with a composition potentially comprising a sigma-1 receptor ligand. Compositions potentially comprising a sigma-1 receptor ligand include, but are not limited to, synthetic combinatorial libraries of small molecule ligands, eukaryotic whole cell lysates or extracts and media conditioned by cultured eukaryotic cells. Separate measurements of the levels of P-glycoprotein expression in the control and test cell are taken, and compared. Reduced levels of P-glycoprotein expression in the test cell indicate compositions that possess tolerance-reducing activity. Multiple rounds of screening with smaller pools can optionally be carried out to isolate one or more agents having tolerance reducing activity (e.g., a sigma-1 receptor ligand).

Measurements of P-glycoprotein expression can be carried out by all methods known in the art, including but not limited to, measurement of mRNA levels by, for example, reverse transcription PCR, or measurement of protein levels by immunohistochemical techniques, such as western blotting.

In yet another embodiment, methods of screening for agents with sigma-1 receptor binding activity are carried out by first obtaining a test agent that is potentially a sigma-1 receptor ligand. Test agents can be obtained from compositions including, but not limited to, synthetic combinatorial libraries of small molecule ligands, eukaryotic whole cell lysates or extracts and media conditioned by cultured eukaryotic cells. Next, the test agent is contacted with a test cell, wherein the test cell expresses the sigma-1 receptor and high levels of P-glycoprotein, under conditions sufficient to bind to a sigma-1 receptor ligand to a sigma-1 receptor. A measurement of P-glycoprotein expression is taken and a determination is made as to whether the agent possesses sigma-1 receptor binding activity based on the agent's ability to reduce P-glycoprotein expression following sigma-1 receptor binding.

Validation of tolerance-reducing or receptor binding activity can comprise, for example, comparing the result obtained in the test cell with and without competition from known sigma-1 receptor antagonists, such as haloperidol. Competing away of tolerance-reducing or receptor binding activity in the presence of antagonists such as haloperidol can further confirm the effect of an agents having, or potentially having, said activity.

In yet another embodiment, methods of screening for compositions having chemotherapeutic tolerance-reducing activity are carried out by first obtaining a control cell that is sensitive to at least one chemotherapeutic agent. Next, a test cell resistant to the same chemotherapeutic agent is obtained. Both cells are treated with the at least one common chemotherapeutic agent(s). The test cell is contacted with a composition potentially comprising a sigma-1 receptor ligand, and separate measurements of the level of chemotherapeutic sensitivity in the control cell and test cell are measured, at which point chemotherapeutic tolerance-reducing activity of the agent can be deduced. Increased chemotherapeutic sensitivity in the test cell(s) can be shown by a corresponding decrease in viability in the control cell(s). Decreased cell viability can be shown by methods known in the art, including, but not limited to cell death (e.g., necrosis or apoptosis), reduced cell proliferation, DNA fragmentation (e.g., detectable by TUNEL assay) and the like.

Screening methods of the present invention can further comprise methods of obtaining and/or generating data related to drug sensitivity, for example, by collecting data relating to compositions potentially comprising sigma-1 receptor ligands in an automated data acquisition system.

The following examples are provided to illustrate, but not limit, the claimed invention.

### EXAMPLES

### Example 1

### Effect of (+)Pentazocine and Sigma Receptor Ligands on P-Glycoprotein mRNA and Protein Levels in Tumor Cell Lines

(+)Pentazocine is a sigma-1 receptor ligand shown herein to reduce P-glycoprotein expression at the mRNA and protein levels in tumor cell lines (e.g., neuroblastoma cell lines and ADX cell lines). This example depicts methods that can be used for 1) studying the role of other sigma-1 receptor ligands in producing a similar effect in neuroblastoma cell lines, and 2) studying the similar effects of (+)pentazocine, or other sigma-1 receptor ligands, on various multidrug resistant tumor cell lines ex vivo.

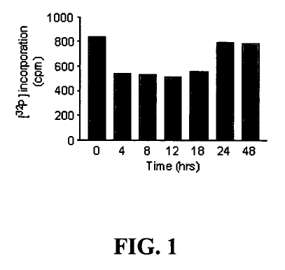

Tests were performed in a BE(2)—C neuroblastoma cell line. This cell line was used to test the ability of (+)pentazocine to reduce levels of Pgp mRNA and corresponding protein expression in a dose-dependent manner. Pgp levels were tested at the mRNA level by reverse-transcription polymerase chain reaction (RT-PCR) in the presence of ³²P-ATP. Incorporation of the radiolabeled nucleotide was measured following electrophoresis of the PCR product. Maximal reduction in Pgp mRNA levels occurred between 12-18 h post-treatment with (+)pentazocine (FIG. 1).

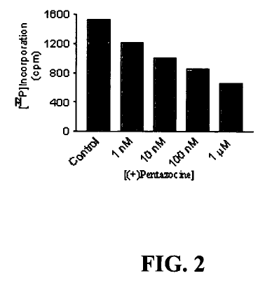

The reduction in mRNA levels was dose-dependent, ranging from concentrations of 1 nM to 11 M (+)pentazocine (FIG. 2). A two-fold decrease in Pgp mRNA was observed following 1 μM administration of (+)pentazocine. Similar effects were observed on Pgp protein levels over a time period ranging from 0-48 hours in BE(2)—C cells as well as ADX cell lines, a derivative of a Chinese hamster lung cell line, DC3-F, that is cross-resistant to a number of chemotherapeutics.

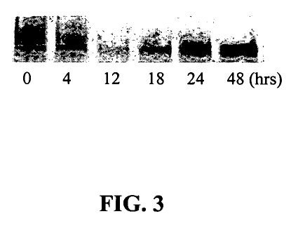

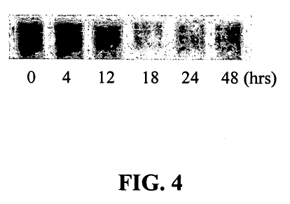

Protein levels were monitored by Western blotting, using monoclonal antibodies specific to Pgp (e.g., C219). Maximal decrease of Pgp protein levels occurred between 12-18 hours in BE(2)—C (FIG. 3) or ADX (FIG. 4) cells, which was analogous to what was observed for Pgp mRNA in the presence of (+)pentazocine.

The following sigma-1 ligands can be administered to the aforementioned neuroblastoma cells: (+)SKF 10047, PRE-084, BD 737 and N-[2-(3,4-dichlorophenyl)ethyl]-N-methyl-2-(1-azapinyl)ethylamine dihydrochloride. Additionally, sigma antagonists such as haloperidol can be screened for their ability to block the effects of (+)pentazocine. An initial dose of 10 μM can be tested, followed by dose-response assays of compounds producing an effect on Pgp levels. mRNA levels can be monitored by RT-PCR in the presence of radiolabeled ATP and protein levels detected by Western blotting.

Additionally, the efficacy of (+)pentazocine treatment on Pgp mRNA and protein levels in other cell lines known to be resistant to chemotherapeutics can be explored. The cell lines used can include ADX cells described above, MES-SA/MX2 cells, which are derived from the human uterine sarcoma cell line MES-SA and resistant to mitoxantrone, and 2780AD cells, which are derived from 2780 human ovarian carcinoma cells and are resistant to doxorubicin and paclitaxel. Cells can be administered varying doses of (+)pentazocine and a time course of action can be utilized to determine optimal activity. As described above, mRNA and protein levels can be monitored using RT-PCR and Western blotting, respectively.

### Example 2

### Effect of (+)Pentazocine Treatment on Multidrug-Resistant Cell Lines

Using the aforementioned drug-resistant cell lines, (+)pentazocine can be assayed for its ability to recapitulate sensitivity to chemotherapeutic agents by measuring 1) cell death and 2) changes in LD₅₀ values of various chemotherapeutic agents.

Cytotoxicity can be measured by trypan blue dye exclusion. The initial LD₅₀ values of the following chemotherapeutic drugs can be determined on parental (sensitive) and resistant cell lines: actinomycin D, doxorubicin, mitoxantrone, vincristine, and paclitaxel. Resistant cells can then be pre-treated with (+)pentazocine, at a concentration previously shown to lower Pgp expression. LD₅₀ values for the aforementioned drugs after (+)pentazocine treatment can be assessed. Repeated administration of (+)pentazocine may be required to sufficiently maintain Pgp downregulation, and if this is the case, (+)pentazocine dosing schedules can be reassessed essentially as described in Example 1.

If changes in LD₅₀ values of the various chemotherapeutic agents are observed in conjunction with (+)pentazocine administration, then a further approach can be undertaken to demonstrate that inhibition of Pgp efflux by (+)pentazocine results in retention of intracellular chemotherapeutic agents. Cells can be treated with a maximal dose of (+)pentazocine and levels of intracellular ³H-vincristine (Amersham Pharmacia) monitored after a 1 hour loading period. The levels of radiolabeled, intracellular vincristine can be compared to cells that were not treated with (+)pentazocine.

### Example 3

### An In Vivo Mouse Model of Restoring Multidrug Sensitivity with (+)Pentazocine

All previous examples were performed, or can be performed, ex vivo. An in vivo model can be used to further demonstrate the clinical relevance of (+)pentazocine and other sigma-1 receptor ligands on Pgp inhibition. If the LD₅₀ values of chemotherapeutic agents outlined in Example 2 are altered after treatment with (+)pentazocine, then the ability of (+)pentazocine to potentiate paclitaxel and doxorubicin-mediated anti-tumor activity can be tested in an in vivo mouse model.

Human ovarian carcinoma xenografts (described in Plumb et al., 1994. Biochem. Pharmacol. 47(2):257-66) can be performed and implanted into athymic Swiss nude mice at a concentration of 2×10⁶ cells in 0.1 ml. Parental cells and resistant cells can be injected into the left and right hind flanks of the mice, respectively. The tumor weight (TW) can be determined twice weekly and calculated by the following formula: TW (mg)=tumor volume (mm³)=d²×D/2, where d and D are the shortest and longest diameters of the tumor, respectively.

Once a TW of 50-100 mg is reached, mice can be randomized into groups of 6-10 and treated with 1) vehicle alone, 2) paclitaxel (36 mg/kg, intravenously) or doxorubicin (5 mg/kg, i.v.) with or without (+)pentazocine (50 mg/kg, s.c.). The chemotherapeutic agents can be optimized according to dosage, based on the amount of tumor cell reduction produced in the parental cell line. Additionally, the timing of administration of (+)pentazocine can be assessed. The reduction in tumors by paclitaxel and doxorubicin can be calculated by % inhibition of tumor weight of control mice, as shown by the following equation: % inhibition=100-(mean TW treated/mean TW control×100). Drug-treated versus control mice can be compared, as well as between mice that were treated with (+)pentazocine versus control mice.

We claim:

1. A method of treating a drug resistant phenotype comprising administering a sigma-1 receptor ligand to a subject in an amount sufficient to restore drug sensitivity.

2. The method of claim 1, wherein the sigma-1 receptor ligand is a (+)enantiomer.

3. The method of claim 1, wherein the sigma-1 receptor ligand is selected from the group consisting of (+)pentazocine, (+)N-allylnormetazocine, 2-(4-morpholinethyl)-1-phenylcyclohexanecarboxylate, cis-N-[2-(3,4-dichlorophenyl)ethyl]-N-methyl-2-(1-pyrrolidinyl)cyclohexylamine, and N-[2-3,4-dichlorophenyl)ethyl]-N-methyl-2-(1-azapinyl)ethylamine dihydrochloride.

4. The method of claim 1, wherein the subject is undergoing a treatment regime with one or more chemotherapeutic agents.

5. The method of claim 4, wherein the chemotherapeutic agent is selected from the group consisting of actinomycin D, doxorubicin, mitoxantrone, paclitaxel and vincristine.

6. The method of claim 4, wherein the chemotherapeutic agent is doxorubicin.

7. The method of claim 4, wherein the chemotherapeutic agent is paclitaxel.

8. The method of claim 4, wherein the chemotherapeutic agent is doxorubicin and the sigma-1 receptor ligand is (+)pentazocine.

9. The method of claim 4, wherein the chemotherapeutic agent is paclitaxel and the sigma-1 receptor ligand is (+)pentazocine.

10. A method for reducing a drug resistant phenotype ex vivo comprising treating a cultured cell with a sigma-1 receptor ligand in an amount sufficient to restore drug sensitivity.

11. The method of claim 10, wherein the cell is undergoing unwanted proliferation.

12. The method of claim 11, wherein the cell is resistant to one or more chemotherapeutic agents.

13. The method of claim 11, wherein the cell is obtained from a tissue source selected from the group consisting of brain, uterine, blood, breast, thyroid, pancreas, gastrointestinal, ovarian, prostate, lung, skin and lymphatic tissue.

14. A method of increasing drug sensitivity comprising administering a sigma-1 receptor ligand to a subject in an amount sufficient to down regulate P-glycoprotein expression in a population of drug resistant cells.

15. A method of attenuating a potentially drug resistant phenotype comprising the steps of:
1) contacting a cell with a sigma-1 receptor ligand
2) contacting a cell with a drug
3) maintaining drug sensitivity in the cell.

16. A method of reducing P-glycoprotein expression in a cell comprising the steps of:
1) contacting a cell with a sigma-1 receptor ligand;
2) binding the sigma-1 receptor ligand to the sigma-1 receptor;
3) reducing P-glycoprotein expression in the cell.

17. A method of screening compositions for tolerance-reducing activity comprising the steps of:
1) contacting a test cell that expresses high levels of P-glycoprotein with a composition potentially comprising a sigma-1 receptor ligand;
2) separately measuring the levels of P-glycoprotein expression in the control cell and test cell; and
3) detecting a reduction in P-glycoprotein expression in the test cell by comparing P-glycoprotein expression in the control cell.

18. The method of claim 17, wherein the composition is selected from the group consisting of synthetic combinatorial libraries of small molecule ligands, eukaryotic whole cell lysates or extracts and media conditioned by cultured eukaryotic cells.

19. A method of screening agents for sigma-1 receptor binding activity comprising the steps of:
1) contacting a potential sigma-1 receptor ligand test agent with a test cell that expresses the sigma-1 receptor and high levels of P-glycoprotein;
2) binding the agent to the sigma-1 receptor; and
3) detecting a reduction in P-glycoprotein expression in the test cell.

20. The method of claim 19, wherein the agent is obtained from a composition selected from the group consisting of synthetic combinatorial libraries of small molecule ligands, eukaryotic whole cell lysates or extracts and media conditioned by cultured eukaryotic cells.

21. A method of obtaining and/or generating data related to drug sensitivity using the method of claim 17.

22. A method of obtaining and/or generating data related to drug sensitivity using the method of claim 17 and an automated data acquisition system.

23. A method of screening compositions for chemotherapeutic tolerance-reducing activity comprising the steps of:
1) treating a control chemotherapeutic-sensitive cell and a chemotherapeutic-resistant test cell with said chemotherapeutic agent;
2) contacting the test cell with a composition potentially comprising a sigma-1 receptor ligand;
3) separately measuring the level of chemotherapeutic sensitivity in the control cell and the test cell; and
4) detecting an increase in sensitivity in the test cell.

24. The method of claim 23, wherein the chemothrapeutic agent is selected from the group consisting of actinomycin D, doxorubicin, mitoxantrone, paclitaxel and vincristine.

25. The method of claim 23, wherein the chemotherapeutic agent is doxorubicin.

26. The method of claim 23, wherein the chemotherapeutic agent is paclitaxel.
